(19) United States
(12) Patent Application Publication
(10) Pub. No.: US 2004/0054379 A1
(43) Pub. Date: Mar. 18, 2004

(54) Surface electrical stimulation for increasing the quality and quantity of synovial fluid in joints

(76) Inventors: William J. Carroll, Sedro Woolley, WA (US); Richard M. Terrell, Vancouver, WA (US)

Correspondence Address: BLANK ROME LLP, 600 NEW HAMPSHIRE AVENUE, N.W., WASHINGTON, DC, 20037, US

(21) Appl. No.: 10/659,278

(22) Filed: Sep. 11, 2003

Related U.S. Application Data
(60) Provisional application No. 60/409,589, filed on Sep. 11, 2002.

(51) Int. Cl. A61N 1/18

(52) U.S. Cl. 607/2; 607/48

(57) ABSTRACT

An electro-medical device and method for improving synovial fluid at a body segment having a joint by applying surface electrical stimulation using surface skin electrodes to the body segment. The electrical stimulation to the body segment and joint may be continuous or in a sequencing pattern. When the electrical stimulation uses a sequencing pattern, it mimics normal electrical sequencing of surrounding muscle of the joint during normal functioning activity. Surface electrical stimulation improves synovial fluid of the body segment and joint and ameliorates degenerative joint disease and osteoarthritis.

Surface Electrical Stimulation (SES) has a beneficial effect on the production and quality of the synovium and the resultant synovial fluid. Electrical stimulation increases blood flow to stimulated areas (U.S. Pat. No. 6,393,328). However, the effect of SES on synovial fluid output and quality and on cartilage and joint deterioration has not been demonstrated or quantified. There is a need to demonstrate that SES has a beneficial effect on decreasing the progressive process of joint deterioration.

Thus, there is a unaddressed need in the industry to demonstrate the beneficial effects of SES for the treatment and ameloriation of osteoarthritis and degenerative joint disease of, but not limited to, the hips, knees, ankles, toes, back, neck, and shoulders.

### SUMMARY OF THE INVENTION

Embodiments of the present invention provide an electro-medical device and method for improving synovial fluid at a body segment having a joint by applying surface electrical stimulation, using surface skin electrodes, to the body segment. The present invention utilizes surface electrical stimulation via surface skin electrodes to pass various types of current across the skin, or transcutaneously, wherein the surface skin electrodes are placed on the surface of the skin. Examples of this type of stimulation include, but are not limited to, Transcutaneous Electrical Never Stimulation (TENS), neuromuscular Electrical Stimulation (NMES), interferential stimulation, diadynamic stimulation, High Volt Galvanic Stimulation (HVGS), Electro-Magnetic and Pulsed Electro-Magnetic Field stimulation (EMF) and (PEMF) and, micro-current stimulation. Those types of surface electrical stimulation can be applied in a continuous manner or they may be applied in patterns of stimulation that mimic the natural functioning of the affected joint.

When mimicking the natural function of the affected joint, normal sequences of stimulation can be used on electrical myographic output of the surrounding musculature. Stimulation that is applied in a continuous manner could range from 0.1 mA to 150 mA as rated into a 500 ohm load. Sequential or pattern type stimulation that mimics the normal action of the effected joint could be in a range from 5 mA to 140 mA as rated into a 500 ohm load. The duration of stimulation would be from about 10 minutes to about 4 hours per day.

Briefly described, in architecture, one embodiment of the invention, among others, can be implemented as follows. An electrical-medical device for improving synovial fluids at a body segment having a joint is described by applying surface electrical stimulation using surface skin electrodes to the body segment. The device generates electrical stimulation to the joints both continuously and in a manner that mimics normal electrical sequencing of surrounding muscles of the joint during normal functioning activity. In another embodiment of the invention, the electrical stimulation of the joint area is by one of the stimulation methods previously described in the disclosure.

Embodiments of the present invention can also be used to provide methods for electrical stimulation of the body segment. In that regard, one embodiment of such a method, among others, can be broadly summarized by the following procedure: A method of improving synovial fluid in a body segment having a joint by applying electrical stimulation using surface skin electrodes to the body segment. The electrical stimulation type can vary as those described previously above in the disclosure. The electrical stimulation can be both continuous and sequential to mimic normal electrical sequencing of surrounding muscles of the joint. It is intended that all of the embodiments are utilized for delaying the onset and improving arthritis and a body sac segment having a joint.

Other systems, methods, features, and advantages of the present invention will be or become apparent to one with skill in the art upon examination of the following drawings and detailed description. It is intended that all such additional systems, methods, features, and advantages be included with this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed clearly upon illustrating the principles of the present invention. Moreover, in the drawings, like reference numerals designate corresponding parts throughout the several views.

FIG. 1 is a drawing illustrating the degradation of the human knee joint due to osteoarthritis with the formation of osteophytes and degeneration of the first protective cartilage in the medial compartment of the right knee;

FIG. 2 is a drawing illustrating the destruction of the joint articular surface in the medial compartment of the left knee;

FIG. 3 is a drawing illustrating an embodiment of the invention for placement of surface skin electrodes to promote electrical stimulation of the entire joint area; and

FIG. 4 is a drawing illustrating an embodiment of the invention for placement of surface skin electrodes to mimic the natural movement pattern of the effected joint.

### DETAILED DESCRIPTION OF THE PREFERRED EMBODIMENT

A preferred embodiment of the invention and modifications thereof will now be described with reference to the drawings.

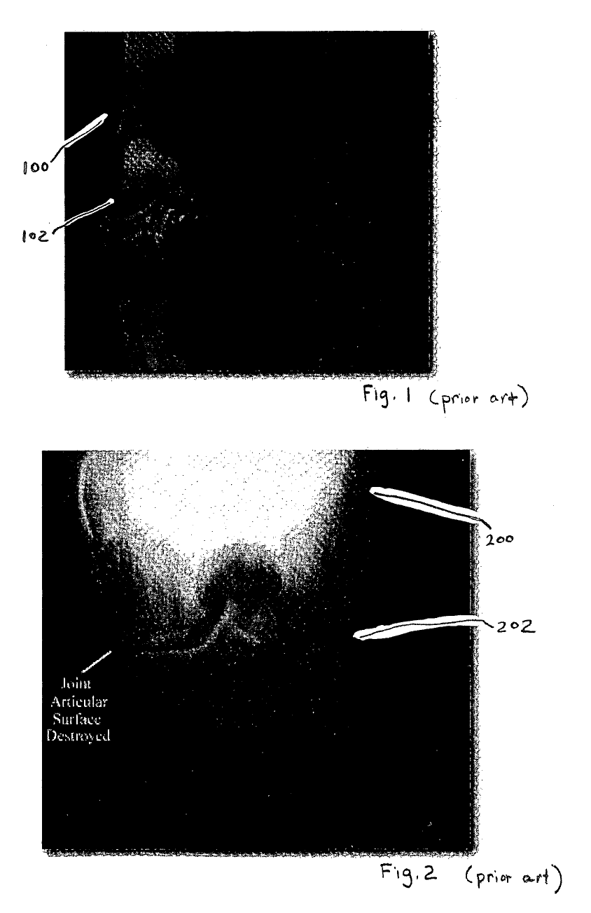

FIG. 1 illustrates the medical compartment of the right knee as the area of joint deterioration/degradation. Another view of the medical aspect of the left knee, the joint area, is shown in FIG. 2

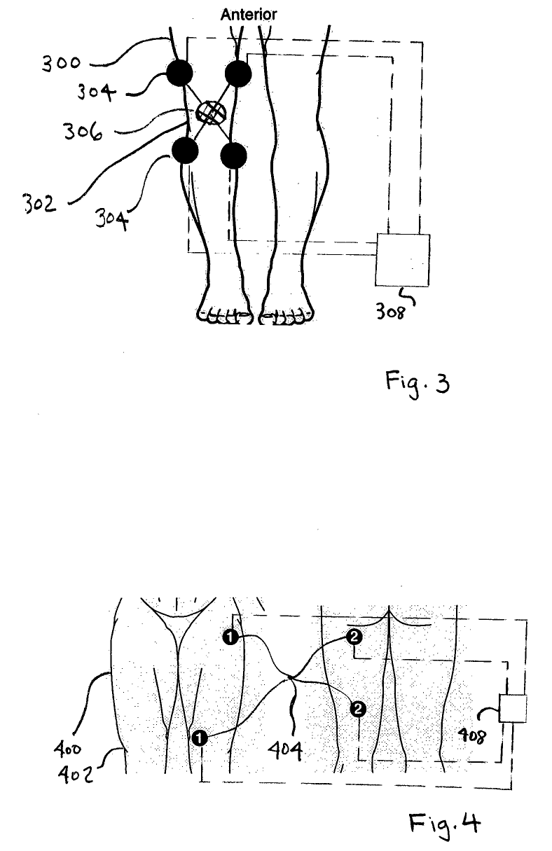

FIG. 3 shows a method of improving synovial fluid in a knee joint 302 by applying electric stimulation using surface skin electrodes 304. The surface skin electrodes 304 are placed in such a manner to surround the affected area both proximally and distally to essentially bathe the area with electrical stimulation. In this embodiment, the electrical stimulation type is interferential because the surface skin electrodes 304 are placed to generate a cross pattern of stimulation 306 for the muscle joint area. The electrical stimulation may be provided by means of an electro-medical device 308 such as the muscle stimulator disclosed in U.S. Pat. No. 6,393,328 to the assignee of the instant application, the disclosure of which is incorporated herein by reference in its entirety. The electrical stimulation provided by the surface skin electrodes 304 is applied in a continuous manner to the joint area in the body segment 300.

In FIG. 4, the claimed invention utilizes the surface skin electrodes 404 to promote electrical stimulation of the surrounding musculature of the joint 402 in the respective body segment 400. The surface skin electrodes 404 are placed at predetermined locations to mimic the natural movement pattern of the affected joint 402. In this embodiment, the electrodes 404 are placed on a front area of the thigh and a back area of the thigh. The electrodes 404 are connected to an electro-medical device 408 for supplying the electrical stimulation signals. With this arrangement, the electrical stimulation can mimic a natural functioning of the affected joint by sequencing stimulation based on the electro-myographic output of the surrounding musculature. This type of stimulation stimulates the affected muscle groups to simulate a pattern of normal joint action and function that would mimic activities of daily living (walking, running) but would not produce destructive wear and tear on the joints that would normally be seen in a weight bearing situation.

In an alternative embodiment, as described above, the electrical stimulation types are varied dependent upon the desired mode of stimulation. Here again, the different types of surface electrical skin stimulation can be applied in a continuous manner or they may be applied in patterns of stimulation that mimic the natural functioning of the affected joint. When stimulation is applied in a continuous manner, it ranges from 0.1 mA to 150 mA as rated into a 500 ohm load. When electrical stimulation is of a sequential or pattern type, thus mimicking the normal action of the affected joint, it would be in a range from 5 mA to 150 mA as rated into a 500 ohm load. In both cases, the duration of stimulation would be from about 10 minutes to about 4 hours per day.

Degenerative processes or wear and tear may cause the aforementioned disorders associated with osteoarthritis and degenerative joint disease. However, it may also result from an injury to the affected body part earlier in life, or there may be a genetic tendency.

It should be emphasized that the above-described embodiments of the present invention, particularly, any “preferred” embodiments are merely possible examples of implementations, merely set forth for a clear understanding on the principles of the invention. Many variations and modifications may be made to the above-described embodiment(s) of the invention without departing substantially from the spirit and principles of the invention. All such modifications and variations are intended to be included herein within the scope of this disclosure and the present invention, and protected by the following claims.

We claim:

1. An electro-medical device for improving synovial fluid at a body segment having a joint by applying surface electrical stimulation to the body segment.

2. The device of claim 1, wherein the electrical stimulation is at least one of transcutaneous electrical nerve stimulation, neuromuscular electrical stimulation, interferential stimulation, diadynamic stimulation, high volt galvanic stimulation, electro-magnetic field stimulation, pulsed electro-magnetic field stimulation, and micro-current stimulation.

3. The device of claim 2, wherein the interferential stimulation generates a current with a resultant beat frequency from 0-250 Hz.

4. The device of claim 2, wherein the interferential stimulation generates an alternating current with a base medium frequency of at least 1 KHz but no more than 20 KHz.

5. The device of claim 1, wherein the electrical stimulation imparts to the body segment at the joint.

6. The device of claim 1, wherein the electrical stimulation is applied continuously.

7. The device of claim 6, wherein the electrical stimulation is within a range from 0.1 mA to 150 mA as rated into a 500 ohm load.

8. The device of claim 1, wherein characteristics and sequencing of the electrical stimulation to the body segment mimics normal electrical sequencing of surrounding muscles of the joint during normal functioning activity.

9. The device of claim 8, wherein the electrical stimulation is within a range from 5 mA to 150 mA as rated into a 500 ohm load.

10. The device of claim 1, wherein the electrical stimulation includes a duration from 10 minutes to 4 hours per day.

11. A method of improving synovial fluid in a body segment having a joint comprising applying electrical stimulation using surface skin electrodes to the body segment.

12. The method of claim 11, wherein the electrical stimulation is at least one of transcutaneous electrical nerve stimulation, neuromuscular electrical stimulation, interferential stimulation, diadynamic stimulation, high volt galvanic stimulation, electro-magnetic field stimulation

13. The method of claim 12, comprising generating an interferential current with a resultant beat frequency of at least 1 KHz but no more than 20 KHz.

14. The method of claim 11, further comprising imparting the electrical stimulation to the body segment at the joint.

15. The method of claim 11, further comprising applying the electrical stimulation continuously.

16. The method of claim 15, wherein the electrical stimulation is within a range from 0.1 mA to 150 mA as rated into a 500 ohm load.

17. The method of claim 11, further comp[rising sequencing the electrical stimulation to the body segment to mimic normal electrical sequencing of surrounding muscles of the joint during normal functioning activity.

18. The method of claim 17, wherein the electrical stimulation is within a range from 5 mA to 150 mA as rated into a 500 ohm load.

19. The method of claim 11, wherein the electrical stimulation includes a duration from 10 minutes to 4 hours per day.

20. An electro-medical device for delaying onset of arthritis at a body segment having a joint by applying surface electrical stimulation using surface skin electrodes to the body segment.

21. The device of claim 20, wherein the electrical stimulation is at least one of transcutaneous electrical nerve stimulation, neuromuscular electrical stimulation, interferential stimulation, diadynamic stimulation, high volt galvanic stimulation, electro-magnetic field stimulation, pulsed electro-magnetic field stimulation, and micro-current stimulation.

22. The device of claim 20, wherein the interferential stimulation generates a current with a resultant beat frequency from 0-250 Hz.

23. The device of claim 22, wherein the interferential stimulation generates an alternating current with a base medium frequency of at least 1 KHz but no more than 20 KHz.

24. The device of claim 20, wherein the electrical stimulation imparts to the body segment at the joint.

25. The device of claim 20, wherein the electrical stimulation is applied continuously.

26. The device of claim 25, wherein the electrical stimulation is within a range from 0.1 mA to 150 mA as rated into a 500 ohm load.

27. The device of claim 20, wherein characteristics and sequencing of the electrical stimulation to the body segment mimics normal electrical sequencing of surrounding muscles of the joint during normal functioning activity.

28. The device of claim 27, wherein the electrical stimulation is within a range from 5 mA to 150 mA as rated into a 500 ohm load.

29. The device of claim 20, wherein the electrical stimulation includes a duration from 10 minutes to 4 hours per day.

30. A method of treating arthritis in a body segment having a joint comprising applying electrical stimulation to the body segment.

31. The method of claim 30, wherein the electrical stimulation is at least one of transcutaneous electrical nerve stimulation, neuromuscular electrical stimulation, interferential stimulation, diadynamic stimulation, high volt galvanic stimulation, electro-magnetic field stimulation

32. The method of claim 30, comprising generating an interferential current with a resultant beat frequency of at least 1 KHz but no more than 20 KHz.

33. The method of claim 30, further comprising imparting the electrical stimulation to the body segment at the joint.

34. The method of claim 30, further comprising applying the electrical stimulation continuously.

35. The method of claim 34, wherein the electrical stimulation is within a range from 0.1 mA to 150 mA as rated into a 500 ohm load.

36. The method of claim 30, further comprising sequencing the electrical stimulation to the body segment to mimic normal electrical sequencing of surrounding muscles of the joint during normal functioning activity.

37. The method of claim 36, wherein the electrical stimulation is within a range from 5 mA to 150 mA as rated into a 500 ohm load.

38. The method of claim 30, wherein the electrical stimulation includes a duration from 10 minutes to 4 hours per day.
